(19) United States
(12) Patent Application Publication
(10) Pub. No.: US 2021/0289765 A1
(43) Pub. Date: Sep. 23, 2021

(54) DEVICES AND METHODS FOR MONITORING AND ELIMINATION OF HONEY BEE PARASITES

(71) Applicant: Combplex Inc., Ithaca, NY (US)

(72) Inventors: Hailey Scofield, Ithaca, NY (US); Nathan Oakes, Ithaca, NY (US)

(21) Appl. No.: 17/337,833

(22) Filed: Jun. 3, 2021

Related U.S. Application Data
(63) Continuation of application No. PCT/US19/64248, filed on Dec. 3, 2019.
(60) Provisional application No. 62/930,925, filed on Nov. 5, 2019.
(60) Provisional application No. 62/774,574, filed on Dec. 3, 2018.

(51) Int. Cl. A01M 1/22; G01S 17/04

(52) U.S. Cl. CPC A01M 1/226; A01M 2200/011; G01S 17/04

(57) ABSTRACT

Automatic detection and elimination of mites through laser exposure is provided. In various embodiments, light is emitted into a target area from at least one light source. Light reflected from a target within the target area is detected by at least one photodetector. A signal is produced proportional to an intensity of the reflected light. A laser pulse is emitted into the target area by a laser emitter when the signal exceeds a predetermined threshold.

### CROSS REFERENCE TO RELATED APPLICATIONS

This application is a continuation of International Application No. PCT/US2019/064248, filed Dec. 3, 2019, which claims the benefit of U.S. Provisional Application No. 62/774,574, filed Dec. 3, 2018, and U.S. Provisional Application No. 62/930,925, filed Nov. 5, 2019, the entireties of which are hereby incorporated by reference.

### BACKGROUND

Embodiments of the present disclosure relate to devices and methods for robust monitoring and treatment of honey bee parasites, and more specifically, to automatic detection and elimination of mites through laser exposure.

### BRIEF SUMMARY

According to embodiments of the present disclosure, devices for monitoring and treatment of bee parasites are provided. A brood frame has a channel therethrough. An image sensor is disposed on the brood frame and is configured to capture images within the channel. A laser emitter is disposed on the brood frame and is configured to emit laser light into the channel. A computing node is operatively coupled to the image sensor and configured to: detect the presence of a bee-borne mite in the captured images; and activate the laser emitter upon detection of a bee-borne mite.

According to embodiments of the present disclosure, methods of and computer program products for monitoring and treatment of bee parasites are provided. Images are captured within a channel by an image sensor. The channel is disposed through a brood frame, and the image sensor is disposed on the brood frame. Presence of a bee-borne mite is detected in the captured images. A laser emitter is activated upon detection of a bee-borne mite. The laser emitter is disposed on the brood frame and configured to emit laser light into the channel.

According to embodiments, devices for monitoring and treatment of bee parasites are provided. A brood frame has a channel therethrough. A plurality of light sources and photodetectors are disposed on the brood frame. The light sources are configured to illuminate the channel, and the photodetectors are configured to detect light within the channel and generate a signal in response to the detected light. A laser emitter is disposed on the brood frame and is configured to emit laser light into the channel when the signal indicates the presence of a mite.

### BRIEF DESCRIPTION OF THE SEVERAL VIEWS OF THE DRAWINGS

FIG. 1 is a bar graph of mite position on bees.

FIGS. 2A-B illustrate the abdomen of Apis mellifera and the preferred attachment site of the Varroa parasite on overwintering hosts.

FIG. 3 illustrates the location of passage through a comb.

FIG. 4 is a schematic view of an exemplary combined mite tracking and control device according to embodiments of the present disclosure.

FIGS. 5A-B illustrate an exemplary frame-embedded mite detector according to embodiments of the present disclosure.

FIG. 6 is a photograph of an assembled device and frame according to embodiments of the present disclosure.

FIGS. 7A-B illustrates an exemplary mite detection and control device according to embodiments of the present disclosure.

FIG. 8 is a schematic view of an exemplary combined mite detection and control device according to embodiments of the present disclosure.

FIGS. 9A-B illustrate an exemplary frame-embedded mite detector according to embodiments of the present disclosure.

FIGS. 10-11 are images of adult Varroa mites after treatment by laser according to embodiments of the present disclosure.

FIG. 12 illustrates a method of monitoring and treatment of bee parasites according to embodiments of the present disclosure.

FIG. 13 illustrates a method of monitoring and treatment of bee parasites according to embodiments of the present disclosure.

FIG. 14 depicts a computing node according to an embodiment of the present disclosure.

### DETAILED DESCRIPTION

Honey bees' critical role in U.S. crop production has come under threat due to unusually high colony losses in the last decade, following declining numbers of managed bees in preceding years. Average annual U.S. honey bee losses have topped 40% in three of the last five years. A growing consensus of honey bee health research indicates that the decline is largely due to the synergistic effects of five key stressors: pesticides, poor nutrition, low genetic diversity, novel pathogens, and parasites. While interconnected, each of these stressors impact bee populations to varying degrees, with the exceptional negative impact of the external parasitic mite Varroa destructor standing out as a chief and urgent concern. Varroa jacobsonii, although similar in appearance, does not pose the same threat. However, it will be apparent that the techniques described herein are applicable to Varroa jacobsonii, as well as to other parasites.

Varroa mites are large (1-1.8 mm long, 1.5-2 mm wide) ectoparasites that feed on the fat bodies of both developing pupae and adult honey bees. Honey bee (Apis cerana and Apis mellifera) colonies around the globe are infected, and the mite can be reliably found in any colony outside the continent of Australia. In addition to the high energetic costs of their feeding behavior, Varroa mites also act as a key vector for extremely harmful honey bee viruses, including RNA viruses such as the deformed wing virus (DWV). The infection and subsequent parasitic disease caused by Varroa mites is called varroosis.

Over the course of a normal season, the mite population of a colony increases 12-fold or more. Individual bees that have been fed on by mites during development experience dramatic reductions in lifespan, as well as poor performance in in-hive and foraging behaviors. Adult bees infested with actively feeding Varroa mites demonstrate poor flight performance, reduced orientation behavior, and poor associated and unassociated learning performance. Furthermore, the viruses transmitted by Varroa mites to developing and adult bees have been shown to have devastating effects on individual bee performance, including a marked reduction on a workers' ability to return home after foraging). If the mite population within a colony is not kept in check, the colony becomes weaker, less effective at pollination and honey production, and will often die before or during the winter season. Once a honey bee colony dies, there is substantial evidence that the mites find their way into other hives in the area, as far as several kilometers away. This is achieved by riding on worker bees that are either abandoning the failing hive or are visiting from another hive to rob its honey stores. Thus, even one colony with a large mite population can pose an infestation risk to all bee colonies within several kilometers.

The Varroa mite can only reproduce in a honey bee colony. Mites reproduce on a 10-day cycle. The female mite enters a honey bee brood cell. As soon as the cell is capped, the Varroa mite lays eggs on the larva. The young mites, typically several females and one male, hatch in about the same time as the young bee develops and leave the cell with the host. When the young bee emerges from the cell after pupation, the Varroa mites also leave and spread to other bees and larvae. Varroa preferentially spreads to nurse bees, which spend more time near the brood. The mite preferentially infests drone cells, allowing the mite to reproduce one more time with the extra three days it takes a drone to emerge as compared to a worker bee.

Tools and techniques for monitoring and controlling Varroa mites are generally labor-intensive, imperfect, and in many cases lead to weakened colonies or honey contamination.

Four techniques may be used for monitoring a colony for the presence of Varroa mites: the sticky board, the sugar shake, the alcohol wash, and the ether roll. The sticky board method requires the installation of a mesh screen on the underside of each hive, and the placement of an oil- or glue-coated board just below the screen. These sticky boards are retrieved 24-48 hours post-placement, and by counting the number of mature mites that have fallen and stuck to the surface, a beekeeper can produce a very rough estimate of the overall mite population. The other three methods require opening each hive, and obtaining a sample of 300 worker bees from the colony's brood nest, where the number of Varroa mites is highest. In the sugar shake method, the sample bees are coated in powdered sugar, and in the alcohol wash the bees are killed with alcohol. In either case, the substance dislodges the mites, and by shaking or rinsing the sample through a screen, the number of mites can be carefully counted. The ether roll requires killing the sample of bees with automotive starter fluid and then counting the number of mites that stick to the inside of a glass jar. These methods all share the qualities of being inexact, labor-intensive, and highly prone to operator error.

Once mites have been quantified, the beekeeper then determines whether or not the mite population is above the treatment threshold. Recommended treatment thresholds have ranged from <1% to 20% infestation. Current guidelines suggest treatment whenever the mite population exceeds 1-3% infestation. Many commercial and hobbyist beekeepers eschew mite monitoring due to either the complicated logistics encountered in large-scale operations or a lack of training and inclination to monitor parasite levels. In either case, the results are the same: mite-riddled colonies that fail to thrive at best, and collapse at worst, and can easily spread parasites to nearby hives.

Treatment, when indicated, involves application of miticidal chemicals. Each miticide has its own delivery method, strict temperature range of effectiveness, risk of honey and/or beeswax contamination, risk of bee mortality, and risk of queen loss. To add to this complexity, many miticides are ineffective against mites while they are inside capped cells of bee brood, meaning that beekeepers must apply multiple doses of such miticides to their hives over the course of weeks or months. Often, application of a miticide may involve modifying the hives or buying specialized equipment. To date, no miticide is 100% effective at killing Varroa mites, and these parasites have been documented evolving rapid resistance (within a year) to synthetic miticides.

In view of the above limitations of alternative approaches, there remains a need in the art for devices and methods for robust monitoring and treatment of honey bee parasites.

Accordingly, the present disclosure provides various hardware monitoring and treatment solution that leverage behaviors of both honey bees and their parasitic Varroa mites. In various embodiments, a camera is installed in a natural choke point in the colony brood nest and computer vision algorithms are applied to register bees with embedded mites. A laser is then activated as the bee walks by, hitting the mite on the bee and killing the mite.

In various embodiments, a plurality of light sources and photodetectors are installed at a natural choke point in the colony brood nest and the reflected light is used to register bees with embedded mites. A laser is then activated as the bee walks by, hitting the mite on the bee and killing the mite.

The laser plus computer vision/photodetector solutions provided herein are a specifically targeted precision approach that has no lasting effect on non-infected honey bees. Unlike alternative chemical solutions, there is no impact on the brood development, no harm to the queen, and no harm inflicted on non-infected workers. Furthermore, there is no honey contamination, and thus these solutions can be used year-round. The infected bee may receive a minor burn or hair loss on her abdominal exoskeleton where the mite was embedded. This does not have any long-term impact on her behavior.

This approach is more cost-effective than chemical treatment methods, particularly in view of time saved, effectiveness at preventing colony collapse, and length of use.

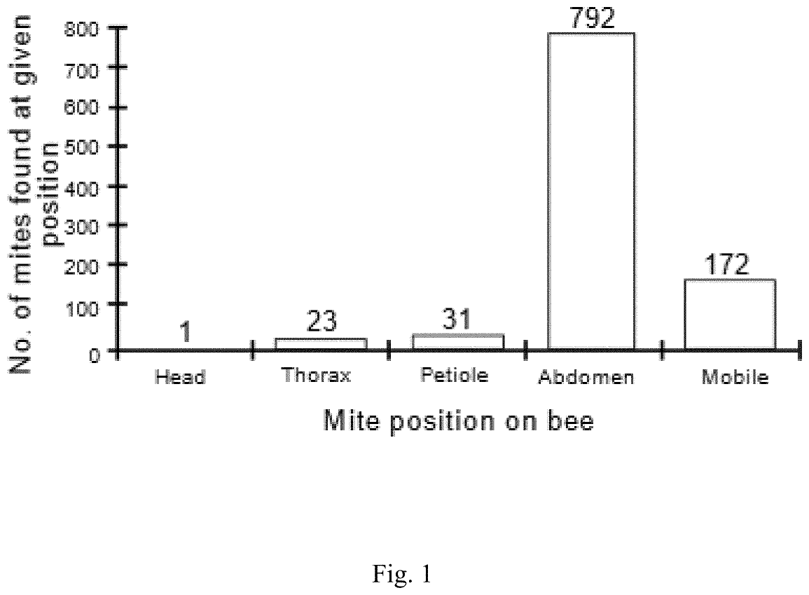

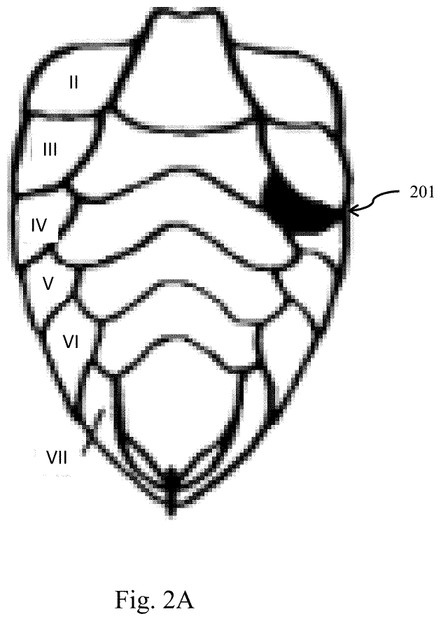

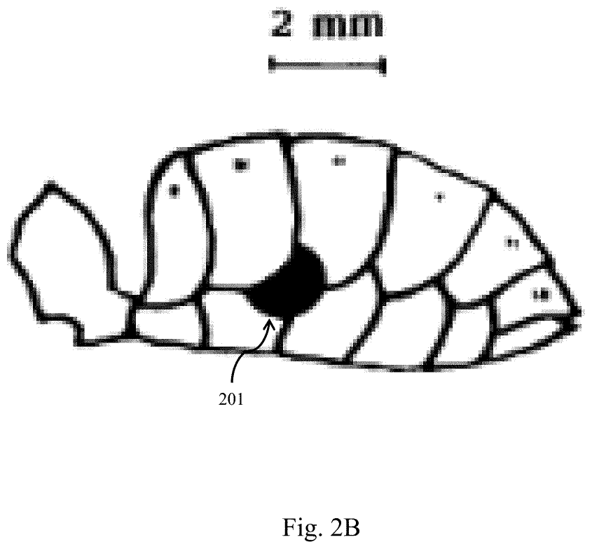

Referring to FIGS. 1-2, the location of Varroa mites on a honey bee is illustrated. As noted above, Varroa mites spend roughly half of their lives on adult bees (the phoretic phase) and half of their lives on developing bee pupae (the reproductive phase) during the summer. On average, mites alternate between approximately 10 days on adult bees, and approximately 10 days reproducing, until the mite dies. During the winter, the mites spend all of their time on adult bees.

FIG. 1 is a bar graph of mite position on bees. In particular, in an exemplary sample of 3458 overwintering Apis mellifera, 1019 mites were located on the head, thorax, petiole, abdomen, or mobile. As shown, the overwhelming number of mites were located on the abdomen of the bees.

FIGS. 2A-B illustrate the abdomen of Apis mellifera and the preferred attachment site of the Varroa parasite on overwintering hosts. FIG. 2A shows the ventral aspect, while FIG. 2B shows the left lateral aspect. The preferred attachment site 201 is located between the left third and fourth tergites. Many mites on adult bees, especially in winter, can be reliably located in the underside of the first few segments of the bee's abdomen, as illustrated.

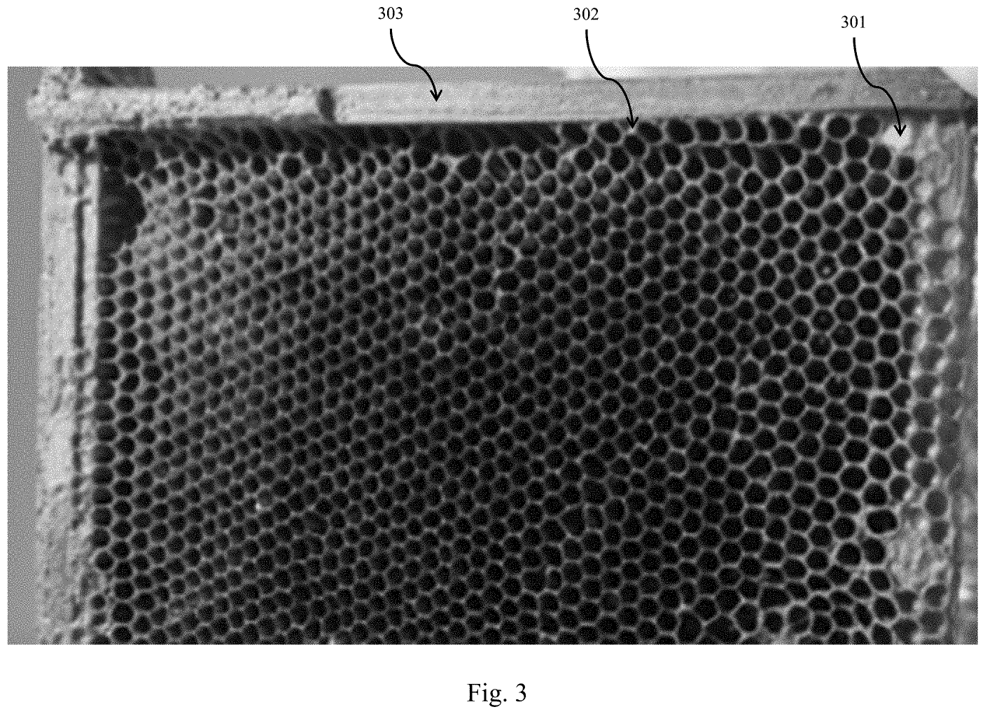

Referring to FIG. 3, the location of passage through a comb is illustrated. Bees in movable frame hives build, or accept pre-constructed, walkways 301 through the corners of combs 302, which allow them to remain continuously in contact with wax comb when moving through and between frames 303 instead of having to walk over wood. These walkways take the form of very small holes in the lower corners of frames. This create a natural chokepoint in bee traffic between frame faces.

Since large numbers of mites can be predictably found in the same locations on bees, and since bees engaged in brood tending regularly pass through frames by use of a natural chokepoint in the hive, detection of Varroa levels in a colony may be achieved by monitoring bee passage through this chokepoint.

In various embodiments, an optical sensor is placed facing the chokepoint used by bees passing through frames. The optical sensor may, for example, be sensitive to infrared light or to visible light. In some embodiments, the optical sensor includes a charge-coupled device (CCD) or active pixel sensors in complementary metal-oxide-semiconductor (CMOS) or N-type metal-oxide-semiconductor (NMOS, Live MOS). However, it will be appreciated that a variety of alternative sensors are suitable for use according to the present disclosure. In some embodiments, a light source is included alongside a camera. For example, as bees cannot see red wavelengths of light (those above 650 nm), introduction of a low-power red light source will not disrupt their natural behavior while providing illumination for the camera. In some embodiments, the light source comprises an LED, which has the advantage of emitting the desired wavelengths without generating disruptive levels of heat.

With a sensor positioned as described above, images of infested and uninfested bees may be captured over a long timescale, resulting a large data set. As set out below, a learning system may then be trained to classify an image as containing an infested or uninfested bee. Once trained, the learning system can be validated against known populations. Moreover, the learning system may be used to determine the overall infestation rate for a given colony in an accurate way.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In exemplary embodiments, a convolutional neural network is used to detect the presence or absence of a mite in a frame of video. In this context, the mite detection problem can be framed as an image classification problem where the class of interest is bee with mite. However, it will be appreciated that mite detection may also be approached as a classification and localization problem, in which the location of a mite within a frame is additionally determined.

A video feed from the image sensor comprises a plurality of sequential frames, which are provided to the convolutional neural network. In some embodiments, the frames originate from the camera at a higher framerate than necessary for the detection task, in which case only a regular subset of the frames is provided to the convolutional network, e.g., every three frames to throttle from 60 fps to 20 fps. A variety of existing convolutional network frameworks may be applied to these problems. In an exemplary embodiments, a plurality of convolution, MaxPool, and ReLU Layers are applied in sequence to each input frame to arrive at class probabilities. In some embodiments, a SoftMax layer is applied to determine a class prediction.

In some embodiments, the CNN or other ANN is provided by a computing node as described below, which comprises software configured to implement the neural network. In some embodiments, the CNN or other ANN is provided by a FPGA or a specialized inference chip. Such embodiments, by virtue of being specialized, have the advantage of lower power consumption and smaller physical footprint.

In various embodiments, a plurality of photodetectors is placed facing the chokepoint used by bees passing through frames. The photodetectors may, for example, be sensitive to infrared light or to visible light. In some embodiments, the photodetectors are sensitive to light in the 600-900 nm range. In some embodiments, the photodetectors have a peak sensitivity to red light. In some embodiments, the peak sensitivity is about 740 nm, for example 740 nm±2%. The photodetectors can be connected to an amplifier circuit, such as a transimpedance amplifier, to amplify the signals obtained from the photodetectors. In some embodiments, the photodetector comprises one or more photodiode, such as a PN photodiode, PIN photodiode, avalanche photodiode, or Schottky photodiode. However, it will be appreciated that a variety of other photodetectors are suitable for use according to the present disclosure, such as photoresistors, phorotransistors, reverse-biased LEDs or pinned photodiodes. Thus, although reference will be made to a photodiode below, other types of photodetectors can be used as well.

In some embodiments, a plurality of light sources is placed facing the chokepoint used by bees passing through frames. In some embodiments, at least one of the light sources is placed alongside a photodetector. In some embodiments, the light sources emit light in the infrared and visible spectrum. In some embodiments, the light sources emit light in the 600-900 nm range. In some embodiments, the light sources emit a red light. In some embodiments, the light sources emit light at about 740 nm, for example 740 nm±2%. A light source that is invisible to bees may be desirable for a variety of reasons. For example, as bees cannot see red wavelengths of light (those above about 650 nm), introduction of a low-power red light source will not disrupt their natural behavior. Bees may notice light within their visible range, and try to investigate.

In some embodiments, the plurality of light sources comprise a light emitting diode (LED), which has the advantage of emitting the desired wavelengths without generating disruptive levels of heat. However, it will be appreciated that a variety of light sources are suitable for use according to the present disclosure, such as fluorescent lamps or organic light-emitting diodes. Thus, although reference will be made to LEDs below, other types of light sources can be used as well. The interior of a colony is generally dark, and the passage is generally heavily shaded. Ambient light is largely filtered out by the passage even when the device is in open air.

Through the use of photodetectors and light sources, the presence of mites can be detected. In some exemplary embodiments, a plurality of photodiodes and LEDs are placed facing the chokepoint used by bees to enter the frame. The LEDs emit a red light, such as light with a wavelength about 740 nm, and the photodiodes have a peak sensitivity at about the wavelength of the LEDs. Each photodetector detects light from the LEDs that is reflected off of surfaces, and generates a voltage in proportion to the amount of detected light. As objects move in front of the LEDs and photodiodes, the light detected by the photodiodes varies. The amount of light reflected by an object depends on the material and color of the surface that the light is incident upon. For example, the more red and shiny an object is, the more red light it will reflect. Because Varroa mites are red, and thus reflect red light, when light shines on the opening in the frame, a different amount of light will be detected when an infested bee enters when compared to a non-infested bee. The resulting voltage difference can be used to detect the presence of a mite. In some embodiments, a mite is detected when the voltage exceeds a threshold value. In some embodiments, a classifier is trained to detect a mite from the set of voltages generated by the photodiodes.

In some embodiments, only one LED and photodiode are used in the detection device. In some embodiments, multiple LEDs and photodiodes are used. In some embodiments, an equal number of LEDs and photodiodes are used. In some embodiments, the LEDs and photodiodes are arranged in a ring. In some embodiments, the LEDs and photodiodes are placed in an alternating fashion, so that each photodiode has an LED on each side, and vice versa. In some embodiments, three photodiodes and three LEDs are used. However, it will be appreciated that a variety of photodiode and LED configurations are suitable for use according to the present disclosure. In some embodiments, the LEDs and photodiodes surround a parasite control device, such as a laser. It will be appreciated that a larger number of photodiodes will increase the precision to which a mite may be located, at the expense of additional cost and complexity.

In some embodiments, detection of a mite occurs when the voltage generated by the photodiodes exceeds a threshold value. The threshold value can be a predetermined value, or it can be learned by a machine learning algorithm. An exemplary formula for determining the voltage generated by a photodetector is given by Equation 1 below. It will be appreciated that a variety of constants (e.g. additive, multiplicative) can be incorporated into Equation 1 according to the present disclosure.

V_{pd}=α*luminance*β*C Equation 1

V_{pd} is the voltage generated by a photodiode. The luminance is the combined luminance of the LEDs, and C is a constant that controls for a variety of factors, such as an amplifier circuit and/or other component-specific variables. α and β are given by Equation 2 and Equation 3 below, respectively:

$$\begin{matrix} α = 1 - \frac{\vert {color}_{m i t e} - {color}_{L E D} \vert}{max ( {color}_{m ite} , {color}_{L E D} )} & Equation 2 \\ β = 1 - \frac{\vert peak\_photodiode \_sensi tivity - col o {r}_{L E D} \vert}{max ( peak\_photodiode \_sensi tivity , col o {r}_{L E D} )} & Equation 3\end{matrix}$$

α is a measure of the similarity between the color of the mite and the color of the LEDs. β is a measure of the similarity between the peak photodiode sensitivity and the color of the LEDs. Both a and are normalized to be within the range 0≤α, β≤1. As can be seen in Equation 2, the closer the color of the mite matches the color of the LED, the more light is reflected, and the closer α is to 1. As can be seen in Equation 3, the closer the peak photodiode sensitivity is to the LED color, the more light is absorbed, and the closer β is to 1. Colorₘᵢₜₑ and color_{LED} are the wavelengths of the mite and the LED colors, respectively, and peak_photodiode_sensitivity is the wavelength for which the photodiode is most sensitive to light.

An exemplary threshold voltage can be calculated by Equation 4 below:

$${V}_{threshold} = 1 - \frac{\vert {λ}_{yellow} - c o l o {r}_{L E D} \vert}{max ( {color}_{m i t e} , c o l o {r}_{L E D} )} * luminance * β * C \qquad Equation 4$$

In Equation 4, color_{LED} is assumed to be above 600 nm, V_{threshold} is the threshold voltage, and λ_{yellow} is the wavelength of yellow light. All other terms are defined as above. It will be observed that Equation 4 is substantially similar to Equation 1, with λ_{yellow} replacing colorₘᵢₜₑ. Thus, if the observed color of a mite is more similar to the LED color than yellow is, the device registers a detection, and a laser can be activated.

Photodiodes with a peak sensitivity wavelength different from the LED wavelength can still be suitable for use according to embodiments of the present disclosure. In some embodiments, the peak sensitivity can differ from the LED wavelength by several percent. Furthermore, in some embodiments, as the detection device must distinguish between yellow and red, the peak sensitivity can have a larger tolerance in the range of frequencies above the LED frequency, as it will not make the photodiode more sensitive to yellow light.

It will be appreciated that as the luminance of the LEDs increases, the threshold increases as well, as there is more light absorbed in the non-infected case. Increasing the luminance can also increase the resolution between colors, until the photodiodes reach their saturation voltage. The threshold voltage can also be affected by a variety of other factors, such as the LED color, the photodiode characteristics, and the amplifier circuit used.

In some embodiments, dividers are placed between the LEDs and the photodiodes in order to reduce the amount of light coming into the photodiodes directly from the LEDs. This improves the resolution of the detector. In some embodiments, the resolution increases as the photodiodes and LEDs are made smaller. In some embodiments, the LEDs and photodiodes are installed as surface mount components. It will be appreciated that the divider size is limited by the focal length of the device. In particular, as the dividers extend further in front of the LEDs and photodiodes, the minimum possible focal length is likewise increased.

In some embodiments, only some (e.g., half or more) of the photodiodes must exceed the threshold voltage to trigger the laser. In some embodiments, all of the photodiodes must exceed the threshold voltage to trigger the laser. In embodiments where the photodiodes are arranged in a ring configuration, when all of the photodiodes exceed the threshold voltage, it is known that there is a red object along the central axis of the array. In some embodiments, a laser diode and lens assembly is located in the center of the array, and is triggered when all of the photodiode thresholds are exceeded.

In some embodiments, the device continuously monitors a passage for a mite. In some embodiments, the device measures the voltages from the photodiodes at discrete intervals (e.g., every few milliseconds). In some embodiments, a motion sensor is placed near the passage and sends a signal to the device when motion is detected. Upon receipt of the signal, the device measures the voltage from each photodetector. In some embodiments, the voltages measured are preprocessed to reduce the effects of noise or to amplify the difference between different readings. In exemplary embodiments, the sensors are measured at a frequency of about 1/100 second. Smoothing may be applied to the input over the past several readings to denoise the signal. It will be appreciated that additional signal processing and denoising techniques may be applied as are known in the art.

In some embodiments, a trained classifier is used to detect the presence of a mite. The classifier can be trained by capturing, for each photodetector, voltage readings of infested and uninfested bees a long timescale, resulting a large data set. As set out below, a learning system may then be trained to classify a particular reading as containing an infested or uninfested bee. Once trained, the learning system can be validated against known populations. Moreover, the learning system may be used to determine the overall infestation rate for a given colony in an accurate way.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In exemplary embodiments, mite detection may also be approached as a classification and localization problem, in which the location of a mite based on a reading is additionally determined.

In some embodiments, the CNN or other ANN is provided by a computing node as described below, which comprises software configured to implement the neural network. In some embodiments, the CNN or other ANN is provided by a FPGA or a specialized inference chip. Such embodiments, by virtue of being specialized, have the advantage of lower power consumption and smaller physical footprint.

In addition, as set out below, the output of the threshold comparison or learning system may also be used to drive a parasite control device, such as a laser. In such embodiments, a laser may be fired when a mite is detected, leading to the injury or death of the mite. As described below, the laser may be configured to kill the mite without causing lasting harm to the bee host. However, in some embodiments, the laser may be configured to kill both the mite and the host.

Accordingly, a variety of configurations are provided herein, including a mite detection device and/or a mite control device.

Devices according to the present disclosure may be configured for monitoring use alone, or for monitoring and mite control use. Although various examples provided herein assume a single choke point, the devices provided herein may also be deployed in a feeding location or other location through which bees pass.

In general, the device may be positioned in any area where bees pass in the brood nest. For example, the device may be placed at the inside or outside of any side of a frame. The device may also be placed on the inside wall of a standard hive body, facing any side of the frame.

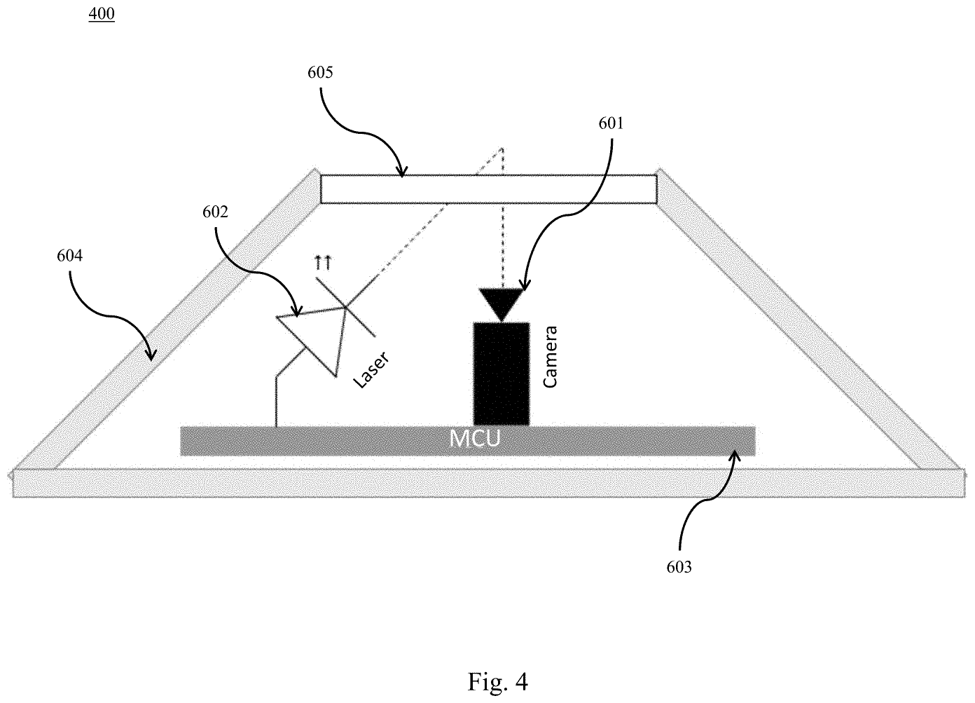

Referring now to FIG. 4, a schematic view is provided of an exemplary combined mite tracking and control device. Device 400 includes camera 401, laser 402, and microcontroller 403. In some embodiments, camera 401, laser 402, and microcontroller 403 are contained in housing 404. In some embodiments, housing 404 is trapezoidal to fit into the corner of a frame (e.g., 303). However, it will be appreciated that a variety of alternative housing shapes may be used. In some embodiments, the housing is integral to a frame, while in some embodiments the housing is connectable to a frame. In some embodiments, the angles of the corners of the housing can range from 45 degrees to fit flush with a rectangular Langstroth frame, or smaller to accommodate the trapezoidal frame of a top bar hive.

In some embodiments, housing 404 includes translucent window 405, configured to face into the corner of a frame when housing 404 is arranged at the corner. Camera 401 faces window 405 and observes honey bees as they walk across the surface to move from one side of a frame to another. This positions the camera to observe the underside of the honey bee as they walk across the window, exposing the area most likely to contain a mite.

The It will be appreciated that the overall length of the device as pictured will determine the distance between window 405 and the interior corner of the frame. A standard tunnel size for a honey bee is ⅜ of an inch in diameter. Accordingly, the device is advantageously positioned such that there is no more than ⅜ of an inch of clearance above window 405. In cases where there is additional clearance, the bees will tend to cover the window with wax in order to obtain their optimal tunnel size. In addition, bees will tend to fill any space less than ¼ inch with propolis. Therefore, a space between ⅜ inch and ¼ inch is in a range of acceptable bee space, with 5/16 inch an average that is generally acceptable. Accordingly, although various embodiments describe a ⅜ inch tunnel, a ¼ inch tunnel or a 5/16 tunnel may be used.

In embodiments where the gap between the window and the frame corner significantly exceeds ⅜ of an inch, a spacer, for example made of molded plastic, may be placed around the space between the frame border and the window of the camera. An opening in the spacer between the front and back side of the frame forms a natural choke point similar to holes naturally created by honey bees in wax foundation frames. The size of this hole is about ⅜ of an inch in diameter. In some embodiments, the length of the opening is also ⅜ of an inch, but may be smaller or wider as well. The opening is positioned no more than ⅜ of an inch off of the window in order to discourage remodeling by the bees.

In embodiments including laser 402, camera 401 and laser 402 may be pre-aligned with window 405. In this way, laser 402 is able to hit a detected mite without mechanical aiming. In other embodiments laser 402 is provided with a gimballed mount, enabling mechanical aiming of the laser by microcontroller 403. In embodiments including laser 402, window 405 is made of a material that is transparent to the wavelength of laser 402. For example, in some embodiments, window 405 comprises glass. In some embodiments, window 405 comprises a corrosion resistant polymer such as polyethylene. It will be appreciated that a variety of polymers may be used, including Polycarbonate (PC), PMMA or Acrylic, Polyethylene Terephthalate (PET), Amorphous Copolyester (PETG), Polyvinyl Chloride (PVC), thermoset Liquid Silicone Rubber (LSR), Cyclic Olefin Copolymer (COC), Polyethylene (PE), Ionomer Resin, Transparent Polypropylene (PP), Fluorinated Ethylene Propylene (FEP), Styrene Methyl Methacrylate (SMMA), Styrene Acrylonitrile Resin (SAN), General Purpose Polystyrene (GPPS), or Methyl Methacrylate Acrylonitrile Butadiene Styrene (MABS).

In some embodiments, laser 402 and/or camera 401 are provided by the terminus of an optical fiber. In such embodiments, the circuitry associated with generating laser light and detecting an image may be located outside of housing 404. For example, a secondary housing may be mounted on the exterior of a hive body, with fiber optic cables leading to housing 404.

In some embodiments, microcontroller 403 receives image data from camera 401, and analyzes it to detect the presence of a bee with a mite. However, in some embodiments, microcontroller transmits image data to a remote computing node for further processing.

As noted above, various algorithms are suitable for recognition of the presence of a mite. For example, supervised learning algorithms may be applied, including: Mahalanobis algorithm; Partial least squares discriminant analysis; Euclidean distance to centroids; linear discriminant analysis; quadratic discriminant analysis; support vector machines; or neural networks.

In some embodiments, when a mite is detected in the path of the laser, the microcontroller activates the laser. In some embodiments, the laser is maintained until the mite is no longer in the field of view of the camera. In some embodiments, the laser is fired for a predetermined period of time.

In an exemplary embodiment, a 0.5 W (500 mW) laser at 450 nm is pulsed for between 1 second and 2 seconds on the mite. However in other embodiments, a 0.25 second pulse or 0.1 second pulse is used at the same power. It will be appreciated that an increase or decrease in power of about 10%, and an increase or decrease in wavelength of about 10% will yield substantially the same results.

In some embodiments, the laser pulse is maintained while the mite continues to be detected in the field of view of the camera. To this end, a neural network may be trained to differentiate between a healthy mite and a mite that has been killed by the laser pulse, for example by identifying exoskeleton damage. In some embodiments, the laser pulse is maintained until a popping sound is detected, indicating that the mite has been exposed to sufficient laser energy to be killed. In such embodiments, a microphone is additionally provided in the casing. In some embodiments, the laser pulse is maintained for a predetermined time.

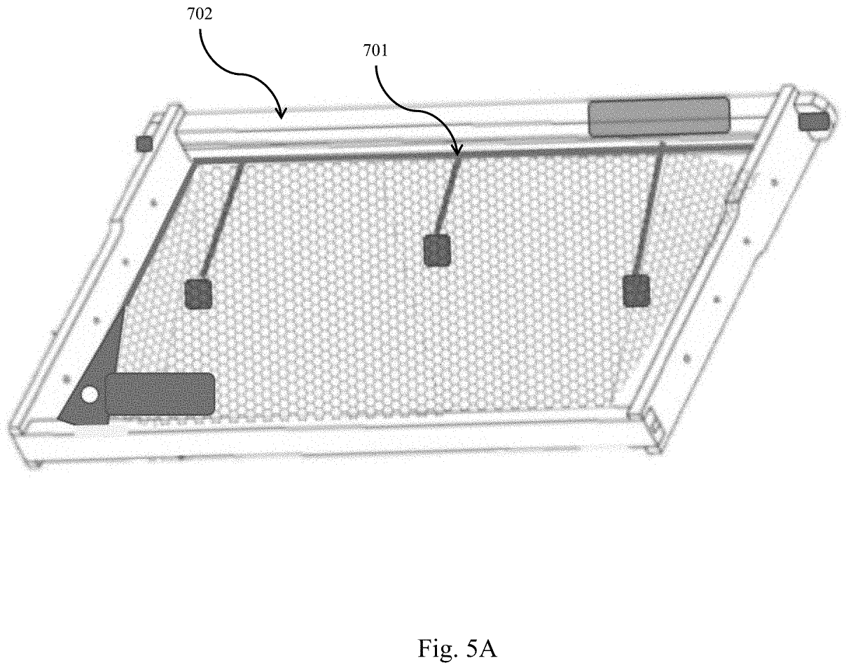

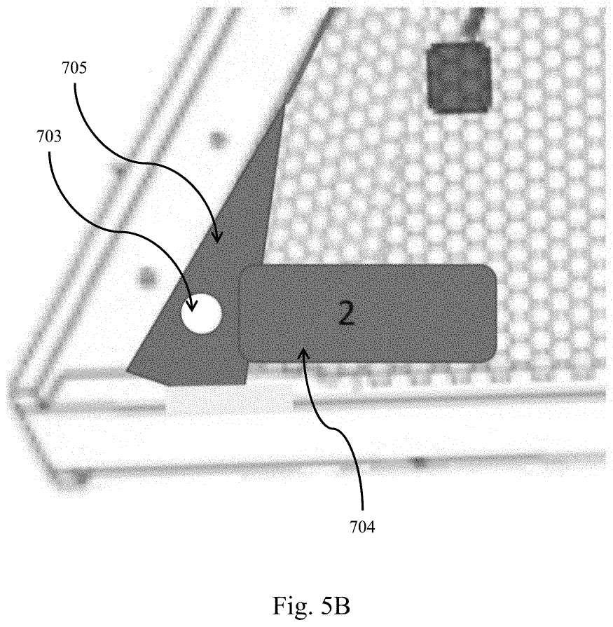

Referring now to FIGS. 5A-B, an exemplary frame-embedded mite detector is illustrated. In various embodiments, an electronic sensor array 501 is into a plastic hive frame 502. In some embodiments, sensor array 501 includes one or more temperature sensor, humidity sensor, Gas/CO₂ sensor, atmospheric pressure sensor, weight sensor, accelerometer, or GPS receiver. In some embodiments, a weight sensor comprises a pressure sensor disposed at one or more ear of a frame, such that the weight of the frame resting on the hive body may be measured. FIG. 5B is a zoomed-in view of the area of frame 502 used to detect mites. In this embodiment, bees can pass through tunnel 503 and be imaged by the camera 504. Spacer 505 is provided to maintain tunnel 503 near camera 504. In some embodiments, tunnel 503 is formed by the presence of housing 404, as described above.

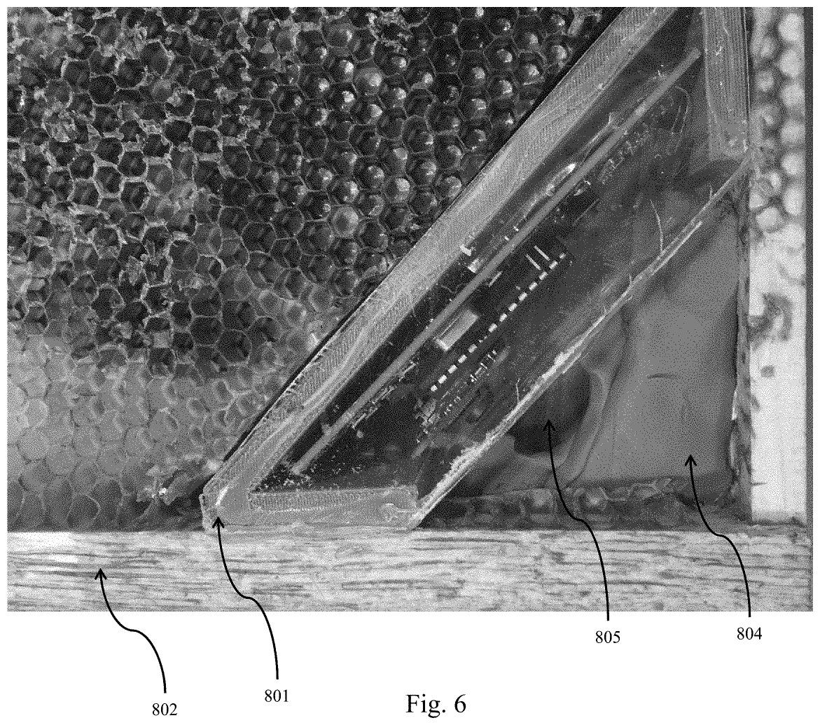

Referring to FIG. 6, a photograph is provided of an assembled device and frame according to embodiments of the present disclosure. In this example, housing 601 (corresponding to housing 404) is arranged at the corner of frame 602. Spacer 604 maintains tunnel 605 in alignment with the camera and laser within housing 601.

Installing electronic equipment into an active beehive is notoriously difficult, since honey is corrosive and bees build their hives according to strict, genetically encoded rules. However, because bees predictably build tunnels at the corners of their combs, the devices described herein leverage the natural choke points while conforming to the bee's natural space.

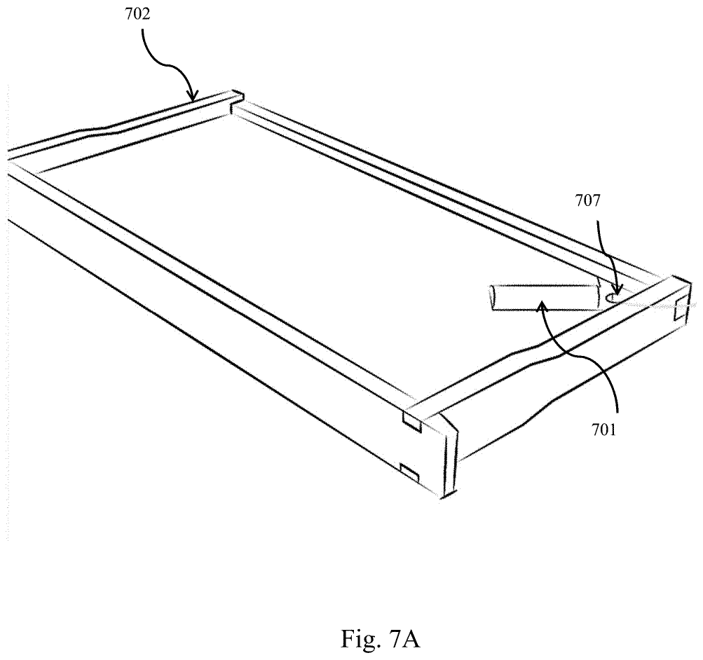

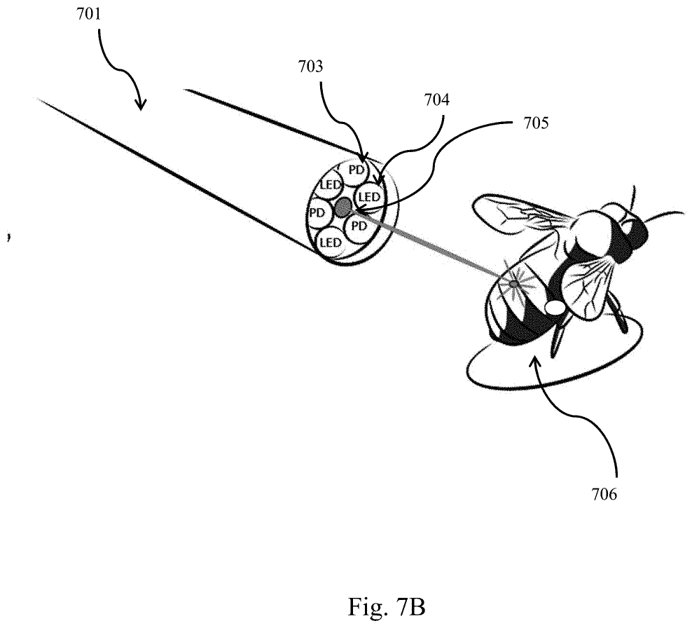

Referring now to FIGS. 7A-B, an exemplary mite detection and control device according to embodiments of the present disclosure is shown. FIG. 7B shows a close-up view of detection device 701 detecting bee 706. Detection device 701 is placed within frame 702, and is directed towards passage 707. Device 701 comprises a plurality of photodiodes 703 and LEDs 704, arranged in a ring around laser 705. Photodiodes 703 and LEDs 704 are arranged alternatingly around the ring, so that each photodetector 703 is between two LEDs 704, and vice versa. When bee 706 passes through passage 707, device 701 detects a mite, and fires laser 705 at the bee.

It will be understood that the orientation and location of the photodiodes, LEDs, and laser relative to the central axis of a passage can depend on the beam angle and the distance between the array and the expected position of the mite. In some embodiments, the LEDs and photodiodes are oriented such that their zenith lines intersect in line with both the laser and the passage, thus exposing the inside of the passage to the laser array.

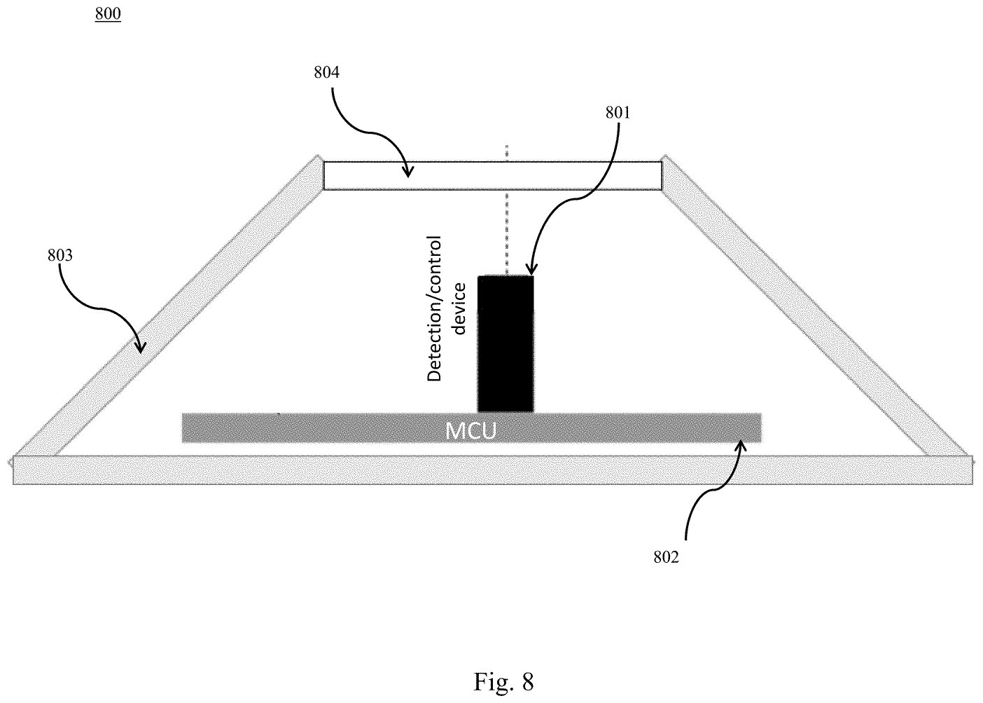

Referring now to FIG. 8, a schematic view is provided of an exemplary combined mite detection and control device. Device 800 includes detection device 801 comprising photodiodes, LEDs, and a laser, and microcontroller 802. In some embodiments, detection/control device 801 and microcontroller 802 are contained in housing 803. In some embodiments, housing 803 is trapezoidal to fit into the corner of a frame (e.g., 303). However, it will be appreciated that a variety of alternative housing shapes may be used. In some embodiments, the housing is integral to a frame, while in some embodiments the housing is connectable to a frame. In some embodiments, the angles of the corners of the housing can range from 45 degrees to fit flush with a rectangular Langstroth frame, or smaller to accommodate the trapezoidal frame of a top bar hive.

In some embodiments, housing 803 and/or detection/control device 801 is easily removed from the frame. In some embodiments, housing 803 and/or detection/control device 801 are formed as a cartridge that can slide in and out of the frame or housing. This allows for easy replacement of components that may have a shorter lifespan than the rest of the device.

In some embodiments, housing 803 includes translucent window 804, configured to face into the corner of a frame when housing 803 is arranged at the corner. Detection/control device 801 faces window 804 and observes honey bees as they walk across the surface to move from one side of a frame to another. This positions the detection/control device to observe the underside of the honey bee as they walk across the window, exposing the area most likely to contain a mite.

It will be appreciated that the overall length of the device as pictured will determine the distance between window 804 and the interior corner of the frame. A standard tunnel size for a honey bee is ⅜ of an inch in diameter. Accordingly, the device is advantageously positioned such that there is no more than ⅜ of an inch of clearance above window 804. In cases where there is additional clearance, the bees will tend to cover the window with wax in order to obtain their optimal tunnel size. In addition, bees will tend to fill any space less than ¼ inch with propolis. Therefore, a space between ⅜ inch and ¼ inch is in a range of acceptable bee space, with 5/16 inch an average that is generally acceptable. Accordingly, although various embodiments describe a ⅜ inch tunnel, a ¼ inch tunnel or a 5/16 tunnel may be used.

In embodiments where the gap between the window and the frame corner significantly exceeds ⅜ of an inch, a spacer, for example made of molded plastic, may be placed around the space between the frame border and the window. An opening in the spacer between the front and back side of the frame forms a natural choke point similar to holes naturally created by honey bees in wax foundation frames. The size of this hole is about ⅜ of an inch in diameter. In some embodiments, the length of the opening is also ⅜ of an inch, but may be smaller or wider as well. The opening is positioned no more than ⅜ of an inch off of the window in order to discourage remodeling by the bees.

In embodiments including a laser, the laser may be pre-aligned with window 804. In this way, the laser is able to hit a detected mite without mechanical aiming. In other embodiments the laser is provided with a gimballed mount, enabling mechanical aiming of the laser by microcontroller 802. In some embodiments, multiple lasers are provided, and each laser is aligned with a different portion of the window 804 and/or the passage that the bees pass through. The lasers can be triggered independently of one another, or they can all fire simultaneously. In embodiments including a laser, window 804 is made of a material that is transparent to the wavelength of the laser. For example, in some embodiments, window 804 comprises glass. In some embodiments, window 804 comprises a corrosion resistant polymer such as polyethylene. It will be appreciated that a variety of polymers may be used, including Polycarbonate (PC), PMMA or Acrylic, Polyethylene Terephthalate (PET), Amorphous Copolyester (PETG), Polyvinyl Chloride (PVC), thermoset Liquid Silicone Rubber (LSR), Cyclic Olefin Copolymer (COC), Polyethylene (PE), Ionomer Resin, Transparent Polypropylene (PP), Fluorinated Ethylene Propylene (FEP), Styrene Methyl Methacrylate (SMMA), Styrene Acrylonitrile Resin (SAN), General Purpose Polystyrene (GPPS), or Methyl Methacrylate Acrylonitrile Butadiene Styrene (MABS).

In some embodiments, the laser, LEDs, or photodiodes are provided by the terminus of an optical fiber. In such embodiments, the circuitry associated with generating laser light and detecting a mite may be located outside of housing 803. For example, a secondary housing may be mounted on the exterior of a hive body, with fiber optic cables leading to housing 803.

In some embodiments, microcontroller 802 receives voltage data from the photodiodes in detection/control device 801, and analyzes it to detect the presence of a bee with a mite. However, in some embodiments, microcontroller 802 transmits voltage data to a remote computing node for further processing.

As noted above, various algorithms are suitable for recognition of the presence of a mite. For example, supervised learning algorithms may be applied, including: Mahalanobis algorithm; Partial least squares discriminant analysis; Euclidean distance to centroids; linear discriminant analysis; quadratic discriminant analysis; support vector machines; or neural networks. In other embodiments, the received voltage data is compared to a threshold value to determine the presence of a mite.

In some embodiments, when a mite is detected in the path of the laser, the microcontroller activates the laser. In some embodiments, the laser is maintained until the mite is no longer detected by the detection/control device. In some embodiments, the laser is fired for a predetermined period of time.

In an exemplary embodiment, a 0.5 W (500 mW) laser at 450 nm is pulsed for between 1 second and 2 seconds on the mite. However in other embodiments, a 0.25 second pulse or 0.1 second pulse is used at the same power. It will be appreciated that an increase or decrease in power of about 10%, and an increase or decrease in wavelength of about 10% will yield substantially the same results.

In some embodiments, the laser pulse is maintained while the mite continues to be detected by the photodiodes. In some embodiments, the laser pulse is maintained until a popping sound is detected, indicating that the mite has been exposed to sufficient laser energy to be killed. In such embodiments, a microphone is additionally provided in the casing.

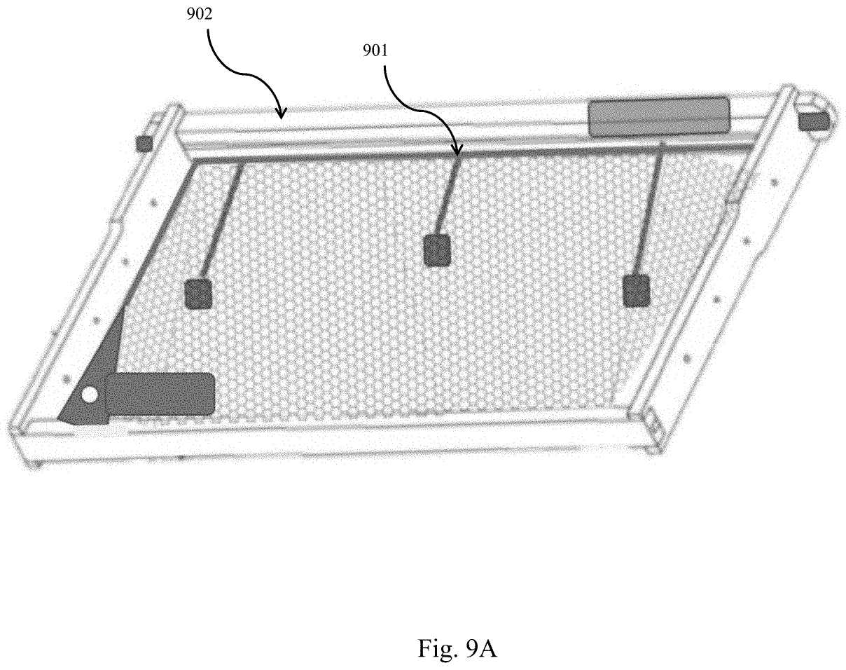

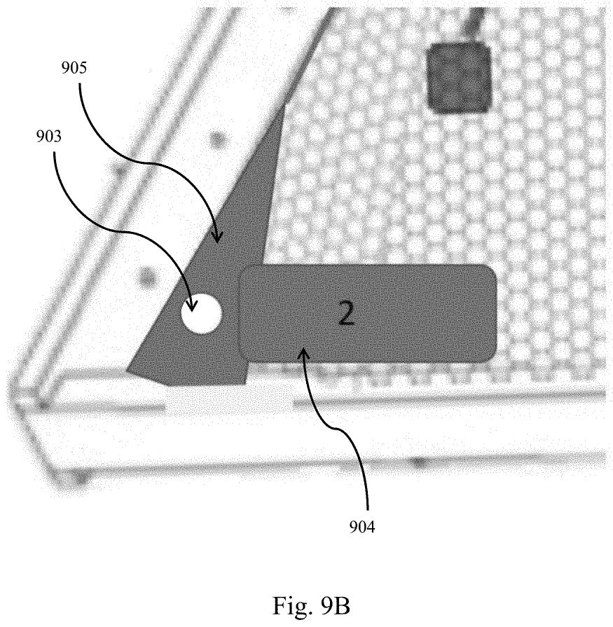

Referring now to FIGS. 9A-B, an exemplary frame-embedded mite detector is illustrated. In various embodiments, an electronic sensor array 901 is included in a plastic hive frame 902. In some embodiments, sensor array 901 includes one or more temperature sensor, humidity sensor, Gas/CO₂ sensor, atmospheric pressure sensor, weight sensor, accelerometer, or GPS receiver. In some embodiments, a weight sensor comprises a pressure sensor disposed at one or more ear of a frame, such that the weight of the frame resting on the hive body may be measured. FIG. 9B is a zoomed-in view of the area of frame 902 used to detect mites. In this embodiment, bees can pass through tunnel 903 and be detected by mite detection and control device 904 comprising a plurality of photodiodes and LEDs. Spacer 905 is provided to maintain tunnel 903 near detection and control device 904. In some embodiments, tunnel 903 is formed by the presence of housing 903, as described above.

Installing electronic equipment into an active beehive is notoriously difficult, since honey is corrosive and bees build their hives according to strict, genetically encoded rules. However, because bees predictably build tunnels at the corners of their combs, the devices described herein leverage the natural choke points while conforming to the bee's natural space.

The photodiode and LED based mite detection methods can be combined with other detection methods, such as those using cameras and digital imagery to detect the presence of a mite. In embodiments where multiple detection methods are used, the lasers can be configured to fire upon the consensus of a predetermined subset of the detection methods used.

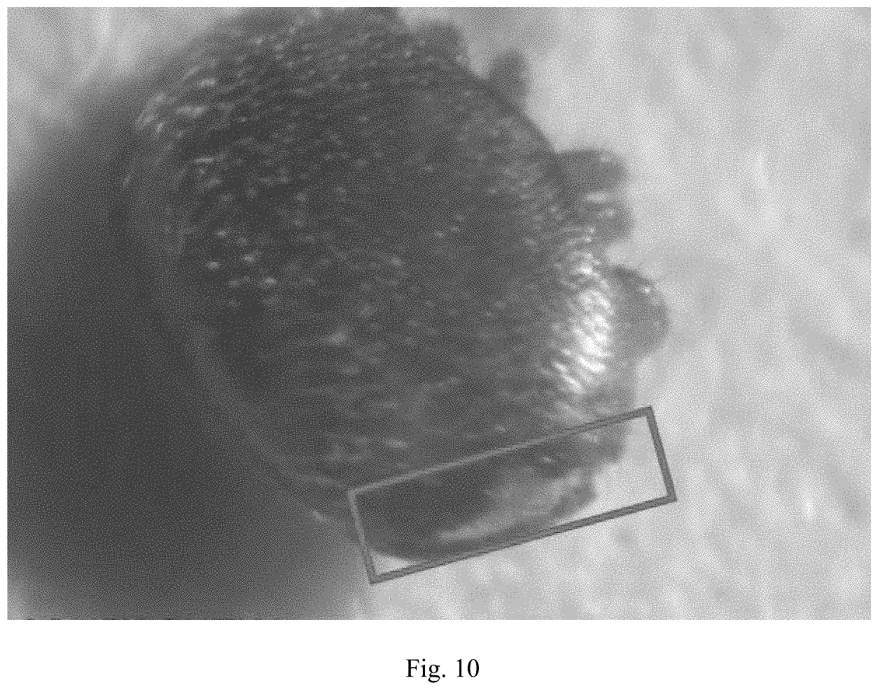

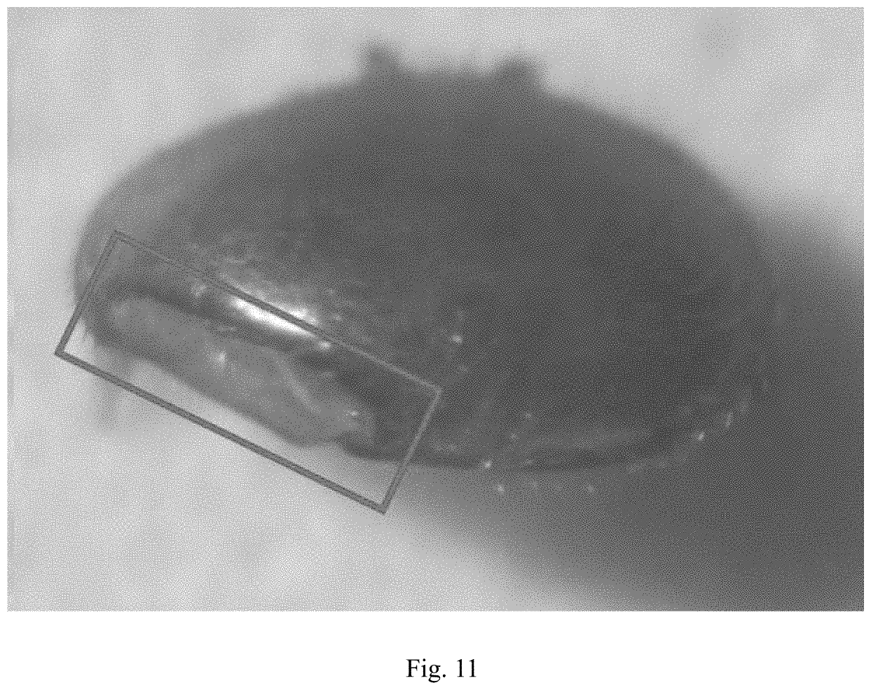

Referring now to FIGS. 10-11, images of adult Varroa mites are provided after treatment by laser. In an exemplary test, a live bees with Varroa mites were targeted with a 0.5 W (500 mW) laser at 450 nm. The mites were actively embedded and feeding from host bee abdomens. After a 0.1 second pulse, catastrophic damage to each mite's exoskeleton was observed (shown in boxes). This led to immediate death of the mites. The bee hosts, although briefly disoriented by the laser pulse, suffered no lasting effects, and the mites dropped off. The laser does not disturb any bees surrounding the target host bee. No behavior is exhibited by the surrounding bees to indicate that the target bee released alarm pheromone when hit. The target bee returned to normal behavior 15 seconds after being hit with a laser pulse of one second. Accordingly, testing indicates that the parasite can be killed with a laser pulse without killing or significantly harming the bee host.

In an exemplary test of a detection and control device according to embodiments of the present disclosure, no false positives were detected by the photodiodes using the threshold-based detection algorithm, the false negative rate was 10% (that is, only 10% of bees with mites were not detected). In this example, 100 bees were considered, all with mites. The field of view of the laser was approximately 0.5 cm in diameter. When a mite was struck by a laser, it was always killed.

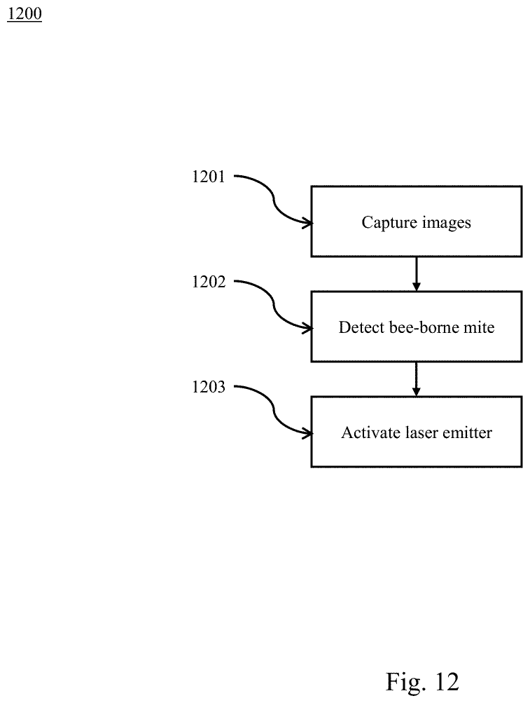

Referring to FIG. 12, a method of monitoring and treatment of bee parasites is illustrated according to embodiments of the present disclosure. At 1201, images are captured within a channel by an image sensor. The channel is disposed through a brood frame, and the image sensor is disposed on the brood frame. At 1202, the presence of a bee-borne mite is detected in the captured images. At 1203, a laser emitter is activated upon detection of a bee-borne mite. The laser emitter is disposed on the brood frame and configured to emit laser light into the channel.

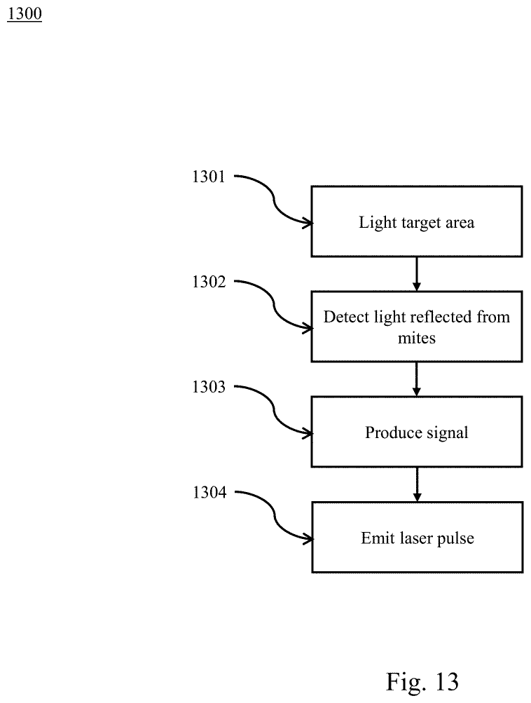

Referring to FIG. 13, a method of monitoring and treatment of bee parasites is illustrated according to embodiments of the present disclosure. At 1301, light is emitted into a target area from at least one light source. At 1302, light reflected from a target within the target area is detected by at least one photodetector. At 1303, a signal is produced proportional to an intensity of the reflected light. At 1304, a laser pulse is emitted into the target area by a laser emitter when the signal exceeds a predetermined threshold.

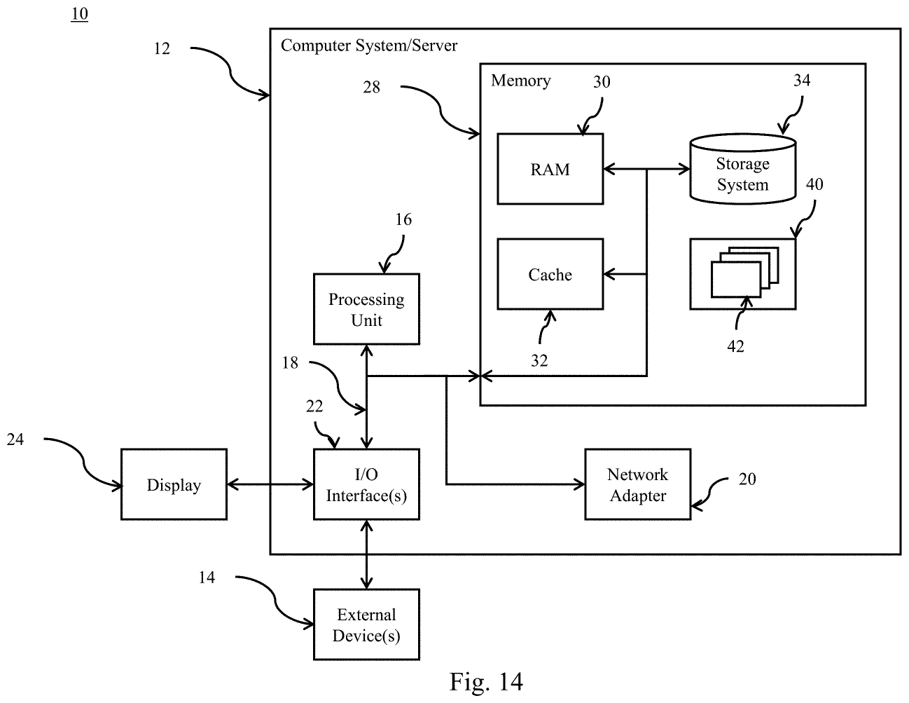

Referring now to FIG. 14, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 14, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device comprising:
at least one light source configured to emit light into a target area;
at least one photodetector configured to detect light reflected from a target within the target area, and to produce a signal proportional to an intensity of the reflected light;
a laser emitter configured to emit a laser pulse into the target area when the signal exceeds a predetermined threshold.

2. The device of claim 1, wherein the target comprises a bee-borne mite.

3. The device of claim 1, wherein
the at least one light source comprises a plurality of light sources, and
the at least one photodetector comprises a plurality of photodetectors.

4. The device of claim 3, wherein the plurality of light sources and the plurality of photodetectors are arranged in a ring.

5. The device of claim 4, wherein the plurality of light sources and the plurality of photodetectors are arranged in an alternating configuration.

6. The device of claim 5, wherein each of the plurality of light sources and each of the plurality of photodetectors are separated by a divider.

7. The device of claim 4, wherein the laser emitter is disposed within the ring.

8. The device of claim 7, wherein each of the laser emitter, the plurality of light sources, and the plurality of photodetectors is focused on a point within the target area.

9. The device of claim 1, wherein the at least one light source comprises an LED.

10. The device of claim 1, wherein the at least one photodetector comprises a photodiode, photoresistor, phototransistor, reverse-biased LED, or pinned photodiode.

11. The device of claim 1, wherein the laser emitter comprises a diode.

12. The device of claim 1, further comprising:
a brood frame having a channel therethrough, the target area being within the channel.

13. The device of claim 12, wherein the channel has a diameter of about ⅜ of an inch.

14. The device of claim 1, wherein the at least one light source is configured to emit light at between 600 nm and 900 nm.

15. The device of claim 1, wherein the at least one photodetector has a peak sensitivity at about 740 nm.

16. The device of claim 1, wherein the laser emitter has a wavelength of about 450 nm.

17. The device of claim 1, wherein the laser emitter has a power of about 0.5 W.

18. The device of claim 1, wherein the laser pulse has a predetermined duration from about 0.1 seconds to about 2 seconds.

19. The device of claim 1, further comprising:
a motion sensor, the at least one photodetector configured to detect light upon detection of motion by the motion sensor.

20. A method comprising:
emit light into a target area from at least one light source;
detecting light reflected from a target within the target area by at least one photodetector;
producing a signal proportional to an intensity of the reflected light;
emitting a laser pulse into the target area by a laser emitter when the signal exceeds a predetermined threshold.
